(19) United States
(12) Patent Application Publication
(10) Pub. No.: US 2008/0090301 A1
(43) Pub. Date: Apr. 17, 2008

(54) Method to detect adulterated specimens

(76) Inventor: Michael Sands Smith, Lawrenceburg, IN (US)

Correspondence Address: William M. Selenke, 5 Drummond St., Cincinnati, OH, 45218, US

(21) Appl. No.: 11/580,769

(22) Filed: Oct. 13, 2006

(51) Int. Cl. G01N 33/00

(52) U.S. Cl. 436/98

(57) ABSTRACT

The invention is a method to detect adulterated specimens submitted for substance abuse. It is concerned with testing a sample of the submitted urine, and in a parallel test analyze a sample of the submitted urine to which has been added known amount of the suspected substance. A control artificial urine with the same concentration of suspected substances as the added known amounts is analyzed in a third parallel test. If the measured amounts of the suspect drug is less than the additive amounts of the submitted urine plus the added know substances the specimen sample is suspect and subject to further consideration.

### FIELD OF INVENTION

The invention is concerned with techniques of indicating adulteration of body fluids such as urine analyzed for substances of abuse in testing laboratories. Such adulteration is used to mask the presence of illegal and abused drugs.

### BACKGROUND OF INVENTION

Prior to employment, professional licensing, or admission to professional schools, an individual may be requested or required to provide a urine sample to be tested. Such tests reveal the presence of illegal drugs or metabolites of such illegal drugs of abuse. An initial or screening test is frequently performed. If positive, the initial test results are usually confirmed by a second more specific method different from that used for initial testing. An initial negative test, however, is usually not confirmed. Thus, an individual who is fearful of a positive result in an initial screening test may alter his or her urine sample to prevent detection of the drug or drug metabolite.

Chemical adulterants may be added to the sample to chemically convert a drug or drug metabolite into a less detectable or undetectable product. Such chemicals include nitrite and chromate. The presence of chemical adulterants is more difficult to assess, since tests for the specific adulterating chemicals must be performed. For example, a group of adulterants has recently been developed to chemically modify 11-nor-delta-9-tetrahydrocannabinol-9-carboxylic acid (delta-9 THC), a metabolite of marijuana. These adulterants prevent recognition of delta-9 THC by drug screening and/or confirmatory assays, with minimal effect on the assay. These adulterants do not alter physical and/or chemical properties commonly monitored to detect adulteration of the sample, such as pH and specific gravity. (See U.S. Pat. No. 5,955,370)

As each new chemical adulterant is recognized and identified, tests are developed for identification of the specific adulterant. However, with the development of multiple adulterants, each of which is chemically distinct and each of which is capable of destroying or masking drugs and/or metabolites, the process of identifying adulterated urine samples becomes increasingly difficult. Multiple tests must be performed on each sample to assure detection of all chemical adulterants. (See U.S. Pat. No. 6,503,726) The present invention presents a method to detect adulterants that reduce the concentration of drugs or metabolites measured in the submitted specimen, compared to the actual biological concentration.

The inventive test system illustrates principles of the present invention for the detection of adulterants. The present test system can detect adulterants of more then one type. That is, the adulterant may destroy or mask the suspect substance so that the suspect substance will not be detected with the standard tests. Such adulterants may be either ingested by the subject of test, or the subject of the test may add adulterants to his urine either after voiding, during voiding, or by substituting a container, which container would have the adulterant in the container proper or in the lid of the container. Current techniques of the art require that the specimen is logged in at the receiving section of the laboratory, and the specimen transferred to a testing station. A subject specimen in a container has a seal for chain of custody documentation. Any number of drugs may be tested from each specimen. However, for the purposes of describing the present inventive process hypothetical drugs R and S will be tested.

### SUMMARY OF THE INVENTION

The invention is method to detect adulterated specimens submitted for substance abuse. It is concerned with testing a sample of the submitted urine, and in parallel test a sample of the submitted urine to which has been added known amount of the suspected substance. A control artificial urine with the same concentration of suspected substances as the added known amounts is analyzed in a third parallel test. If the measured amounts of the suspect drug is less than the additive amounts of the submitted urine plus the added know substances the specimen sample is suspect and subject to further consideration. In more detail the process comprises the steps of accepting the body fluid. The body fluid is divided into one or more aliquots. A first aliquot is processed via standard tests. Prior to processing a second aliquot a standard combination of suspected drugs at known amount(s), greater than the limit of quantitation are added to said aliquot and mixed to ensure uniformity. The second aliquot is set aside under laboratory conditions for a specified time. At the end of the specified time the second aliquots is processed via the same standard means as the first aliquot and the results of the first aliquot, and second aliquot are compared with know amounts of substances in artificial urine to determine if the submitted sample had added adulterants.

### BRIEF DESCRIPTION OF FIGURES

FIG. 1 show that the submitted subject body fluid sample is portioned into aliquot A and aliquot B which are removed for testing.

FIG. 2 illustrates test results of a urine specimen from a negative test subject, which test subject had not used masking substances.

FIG. 3 shows positive subject body fluids sample results for Aliquot A test run.

FIG. 4 demonstrates the absorption of the typical positive control amounts of R and S in artificial urine.

FIG. 5 would indicate a positive test with no adulterants present.

FIG. 6 is an absorption curve of the same urine as shown in FIG. 5 with added adulterants which alters the measured amounts of R and S.

FIG. 7 shows the how the present invention would detect the adulterants as shown in FIG. 6.

### DETAILED DESCRIPTION OF FIGURES

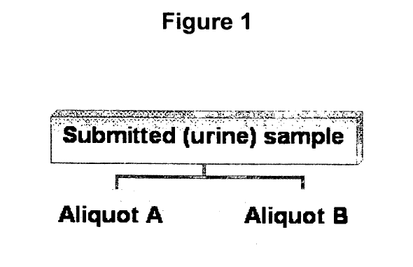

FIG. 1 show that the submitted subject sample providing aliquot A and aliquot B. Aliquot A is essentially the original test sample as is now processed in the current techniques. The present invention requires the processing of an additional aliquot, called Aliquot B. Aliquot B is prepared by adding known concentrations of drugs or metabolites R and S to an aliquot of urine from specimen container. After a specified incubation time and ensuring the aliquot is uniformly mixed, the Aliquot B is analyzed. That is, standard combination of suspected drugs at known amount(s), greater than the limit of quantitation are added to the aliquot and mixed to ensure uniformity;

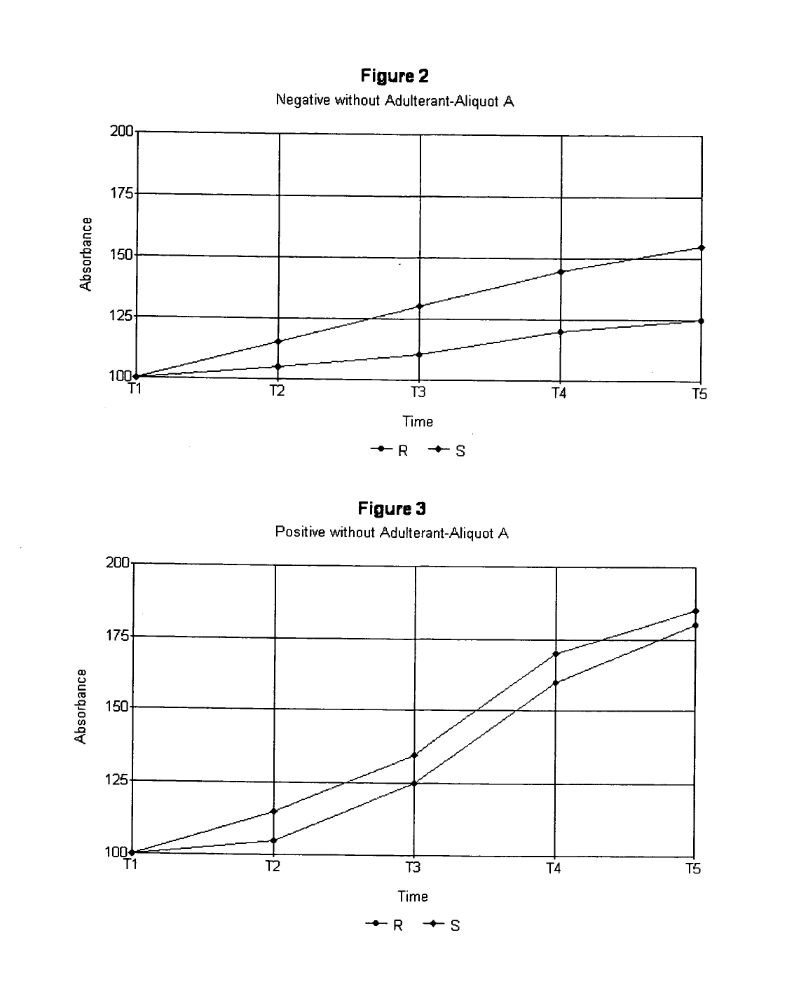

FIG. 2 illustrates urine drug analysis of hypothetical drugs R and S as aliquot A. FIG. 2 shows results of Aliquot A, generally negative test with no evidence of adulterants. There are various analytic techniques for various drugs. The process of the present invention is not dependant on any particular analytic assay. In this hypothetical example of a screening EIA test, the positive cutoff is defined as a change of absorbance above 50 units at specific time points (T3 to T4) on X axis. The graph show absorbance units of 50 to 200 on Y axis and T1 to T5 on the X axis. It is a feature of certain tests that the baseline changes over time. Note that while the absorption units from about 110 to about 125 for R between T3 and T5 and S changed from about 130 to about 155. These are less than the test positive threshold of 50 absorption units.

FIG. 3 shows positive subject body fluids sample results for Aliquot A test run. In this hypothetical example of a screening EIA test, a change of absorbance units from 115 to 155 on Y axis as measured at specific time points (T3 to T4) on X axis. Note, R absorption units increases from about 125 to about 180 and S increases from about 133 to about 185. Note an increase of 50 absorption units indicates a the positive results.

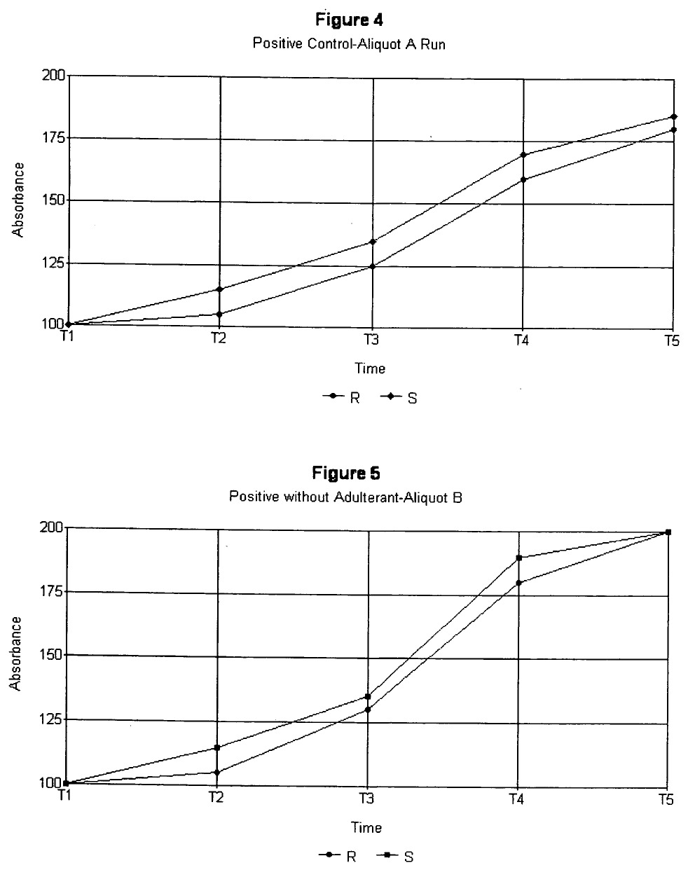

FIG. 4 shows standard positive test controls for known levels of R and S in an artificial urine matrix. It is standard procedure that positive and negative control substances (artificial urine for example) are used to confirm test accuracy. In this hypothetical example of a screening EIA test, a change of absorbance units for substance R from about 125 to 180 on Y axis as measured at specific time points (T3 to T4) on X axis. Likewise, a change of absorbance units for substance S from about 135 to 185 on Y axis as measured at specific time points (T3 to T4) on X axis.

FIG. 5 would indicate a positive test with no adulterants present. The present invention requires the processing of an additional aliquot, called Aliquot B. (See FIG. 1.) Aliquot B is prepared by adding known concentrations of drugs or metabolites R and S to an aliquot of urine from the specimen container. The concentration used for the spiked aliquot B must be greater than the limit of quantitation, but may be less than the cutoff concentration. The expected change in absorbance units at the measurement interval is based on the concentration used. This example uses a cutoff concentration, which is expected to produce a change of >25 absorbance units at T3-T4. That is Aliquot B equals measured sample of urine from subject plus added known concentration of substances R and S. After a specified incubation time and ensuring the aliquot is uniformly mixed, Aliquot B is analyzed. A change of absorbance units for substance R from about 130 to 200 on Y axis as measured at specific time points (T3 to T4) on X axis. Likewise, a change of absorbance units for substance S from about 135 to 200 on Y axis as measured at specific time points (T3 to T4) on X axis. Aliquot A initially tested positive as shown in FIG. 3. Aliquot B, if positive, as shown in FIG. 5, would indicate that there were no adulterants present. The change in absorbance over time is proportional to the quantity of R and S that is added to the aliquot, as demonstrated by positive control test results in FIG. 4.

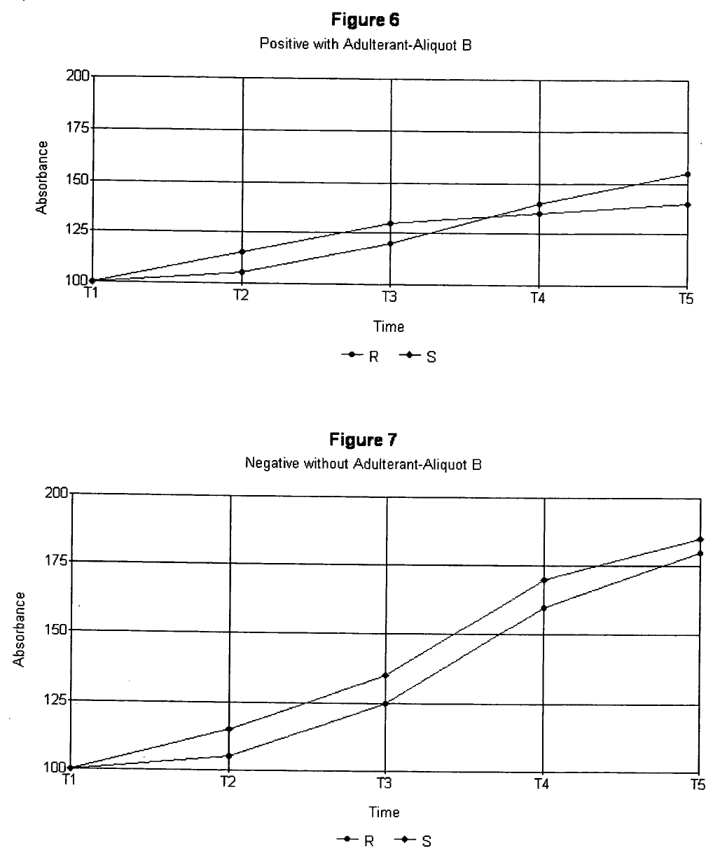

FIG. 6 indicates the presence of adulterant(s) in Aliquot B, resulting in a negative drug test. The measured amount is compared to the expected concentration of the drug spiked aliquot. A change of absorbance units for substance R from about 120 to 155 on Y axis as measured at specific time points (T3 to T4) on X axis. Likewise, a change of absorbance units for substance S from about 130 to 140 on Y axis as measured at specific time points (T3 to T4) on X axis. The figure shows that the drug R is less well blocked than drug S.

FIG. 7 indicates the expected results when the urine is both drug negative and has no added adulterants. A change of absorbance units for substance R from about 125 to 180 on Y axis as measured at specific time points (T3 to T4) on X axis. Likewise, a change of absorbance units for substance S from about 135 to 190 on Y axis as measured at specific time points (T3 to T4) on X axis. As noted, known amounts of drugs R and S are added to “clean” urine with no adulterants resulting in a positive test result the increase in absorption from 115 to 155 or 50 absorption units in FIG. 4.

To summarize: Is the submitted urine specimen has adulterants that can mask or destroy the substances of interest that the client should show in his urine, those added adulterants should destroy or lessen the amounts of known substances. If client has an measured samples of a substance at a acceptable level, the by adding known amounts of that substance to samples of the submitted urine, the amounts of added substance should be measures as the baseline plus the added amounts. If not, the urine sample is suspect. The process of the present invention will determine if the test subject would be subject to rigorous further testing.

I claim:

1) A process to detect drug masking adulterants in body fluids comprising the steps of:
accepting the body fluid;
said body fluid is divided into one or more aliquots;
a first aliquot is processed via standard means;
prior to processing a second aliquot a standard combination of suspected drugs at known amount(s), greater than the limit of quantitation are added to said aliquot and mixed to ensure uniformity;
said second aliquot is set aside under laboratory conditions for a specified time;
at the end of said specified time said second aliquots is processed via the same standard means as said first aliquot;
and the results of the first aliquot, and said second aliquot are compared are compared with known test amounts of substances in artificial urine to determine if the submitted sample had added adulterants.
